(19) United States
(12) Patent Application Publication
(10) Pub. No.: US 2006/0003028 A1
(43) Pub. Date: Jan. 5, 2006

(54) Stable oxidizing bromine composition, method of manufacture and use thereof for biofouling control

(76) Inventors: Craig Myers, Lisle, IL (US); Shunong Yang, Naperville, IL (US)

Correspondence Address: NALCO COMPANY, 1601 W. DIEHL ROAD, NAPERVILLE, IL, 60563-1198, US

(21) Appl. No.: 10/880,628

(22) Filed: Jun. 30, 2004

(51) Int. Cl. A61K 33/00

(52) U.S. Cl. 424/723

(57) ABSTRACT

A stable oxidizing bromine biocidal composition comprising at least one stable oxidizing bromine compound that is prepared from at least one oxidizing chemical reagent, at least one bromide source and at least one bromine or halogen stabilizer, wherein the molar ratio of bromide source to stable oxidizing bromine compound is at least about 2.1, methods of making the composition and methods of using the composition as a biocide in aqueous systems.

### TECHNICAL FIELD

This invention relates to formulations used in biofouling control in industrial water systems. More specifically, this invention relates to an improved stable oxidizing bromine biocidal composition comprising excess bromide, methods of preparing the composition and use of the composition in biofouling control in industrial water systems.

### BACKGROUND OF THE INVENTION

While elemental liquid bromine is an effective biocide, its low solubility (<4 g/100 g water), low boiling point (54.3° C.), high vapor pressure (214 mm Hg at 25° C.) and extreme corrosivity limit its use as a biocide in industrial applications. Another oxidizing bromine compound, bromine chloride, has slightly higher water solubility but is more volatile than elemental bromine. One other oxidizing bromine compound, bromate, is very toxic to mammals and is a suspected carcinogen. Nonoxidizing inorganic bromine compounds, such as bromide, have little or no antimicrobial activity.

A mixture of an aqueous bromine solution and a bromine stabilizer has been used to generate stable oxidizing bromine compounds for use as a biocide. An unstabilized aqueous bromine solution is very acidic, unstable and emits very pungent bromine fumes.

It has also been suggested that an oxidizer, such as hypochlorite, be added to activate the bromide to hypobromite. After the completion of the conversion of bromide to hypobromite, the hypobromite is stabilized by the addition of a halogen stabilizer, such as sulfamate. See, for example, U.S. Pat. Nos. 5,683,654, 6,068,861, 6,156,229, 6,270,722, 6,423,267 and 6,669,904. While these are improved processes which result in a stable concentrated oxidizing bromine biocidal composition, excessive use of halogen stabilizer can result in biocidal performance reduction at use conditions.

Therefore, there is an ongoing need for methods of generating and maintaining higher concentrations of stable oxidizing bromine species and improving their biocidal performance.

### SUMMARY OF THE INVENTION

We have discovered that adding additional bromide to an aqueous stabilized oxidizing bromine biocidal composition results in a composition having enhanced biocidal performance compared to existing stabilized bromine biocidal compositions.

Accordingly, this invention is a stable oxidizing bromine biocidal composition comprising at least one stable oxidizing bromine compound that is prepared from at least one oxidizing chemical reagent, at least one bromide source and at least one bromine or halogen stabilizer, wherein the ratio of bromide source to stable oxidizing bromine compound is at least about 2.1.

### DETAILED DESCRIPTION OF THE INVENTION

The oxidizing bromine biocidal composition of this invention is prepared by admixing at least one oxidizing chemical reagent, at least one bromine or halogen stabilizer and a sufficient excess of least one bromide source to result in a composition having a molar ratio of bromide source to stable oxidizing bromine compound of at least about 2.1. Excess bromide source may be used initially in the preparation of the composition, or alternatively, excess bromide source may be added to the finished composition or to a system being treated with a conventional stable oxidizing bromide composition.

In a preferred aspect of this invention, the bromine or halogen stabilizers are selected from the group consisting of compounds of formula R—NH—R¹, wherein R and R¹ are selected from the group consisting of R²CO, R²SO₂, R²CF₂, R²CHF, H, OH and PO(OH)₂, or a salt thereof, and R² is an alkyl group or an aromatic group, and mixtures thereof.

In another preferred aspect, the oxidizing chemical reagents are selected from the group consisting of alkali and alkaline earth metal hypobromite, alkaline and alkaline earth metal bromates, chlorine gas, hypochlorous acid, alkali and alkaline earth metal hypochlorites, chlorite, hydrogen peroxide, persulfate, permanganate, peracetic acid, bromine, bromine chloride and bromate.

In another preferred aspect, the bromide sources are selected from the group consisting of bromine, alkali and alkaline earth metal bromides, alkali and alkaline earth metal bromates, hydrobromic acid, bromine chloride, and mixtures thereof.

In another preferred aspect, the molar ratio of bromide source to stable oxidizing bromine compound is from about 2.5 to about 5.

In another preferred aspect, the stabilizer is selected from the group consisting of saccharine, urea, thiourea, creatinine, cyanuric acids, alkyl hydantoins, monoethanolamine, diethanolamine, organic sulfonamides, biuret, sulfamic acid, organic sulfamates, melamine and ammonia.

In another preferred aspect, the molar ratio of bromide source to stable oxidizing bromine compound is from about 2.5 to about 3.

The oxidants, bromide sources and halogen stabilizers used to prepare the oxidizing bromine biocidal composition of this invention are known in the art. The ratios of the components, order of addition, pH, temperature and other variables are selected to result in a composition having the desired properties. Several illustrative preparations are described below.

In an embodiment, the oxidizing bromine biocidal composition is prepared by mixing an alkali or alkaline earth metal bromide and an alkali or alkaline earth metal bromate in water to provide an aqueous solution, cooling the solution to a temperature of less than 25° C., preferably less than 20° C. and more preferably less than 10° C., and thereafter adding a halogen stabilizer to the solution.

In this embodiment, the alkali or alkaline earth metal bromide and the alkali or alkaline earth metal bromate are preferably added in a molar ratio of at least about 5.3:1 to result in a composition having the desired excess of bromide source. The molar ratio of the halogen stabilizer to the oxidizing bromine is preferably close to 1.

In this embodiment, the step of adding the halogen stabilizer results in the solution having a pH of less than about 2.

In this embodiment, the method comprises agitating the solution for a time period of greater than about 5 minutes after the step of adding the halogen stabilizer. In this embodiment, the method further comprises adjusting the solution to a pH of greater than 13 through the addition of alkali or alkaline earth metal hydroxide after the step of adding the halogen stabilizer if the product is to be stored for an extended period prior to use.

In another embodiment, the stable oxidizing bromine solution is prepared by combining a bromine source and a stabilizer to form a mixture and then adding an oxidizer to the mixture. If the composition is to be stored for an extended period an alkaline source may be added to adjust the pH of the mixture to at least 13.

Suitable bromine sources in this embodiment include hydrobromic acid, bromine chloride, elemental bromine and alkali or alkaline earth metal bromides, such as sodium bromide, potassium bromide and lithium bromide.

Suitable stabilizers in this embodiment are as described above. Sulfamic acid is preferred.

Suitable oxidizers in this embodiment include chlorine gas, hypochlorous acid, hypochlorite salt, chlorite, chlorate, elemental bromine, bromine chloride, hydrogen peroxide, persulfate, permanganate and peracetic acid. It is believed that other peroxy compounds can also be used in accordance with this embodiment.

In this embodiment, the alkaline source is preferably an alkali or alkaline earth metal hydroxide. Suitable alkaline sources include sodium hydroxide, lithium hydroxide, potassium hydroxide, magnesium hydroxide and calcium hydroxide.

In this embodiment, the reaction is maintained at a temperature of less than about 80° F., and preferably in the range of about 40 to about 70° F. This embodiment can be carried out as either a batch or continuous process.

In this embodiment, the molar ratio of the bromine source to the oxidizer is preferably at least about 2.1 in order to result in a composition having the desired excess of bromide source. The molar ratio of the halogen stabilizer to the oxidizing bromine is preferably about 1.

In another embodiment, the stable oxidizing bromine biocidal composition is prepared by:
a. Mixing an aqueous solution of alkali or alkaline earth metal hypochlorite with a water soluble bromide ion source;
b. Allowing the bromide ion source and the alkali or alkaline earth metal hypochlorite to react to form a 0.5 to 30 percent by weight aqueous solution of unstabilized alkali or alkaline earth metal hypobromite;
c. Adding to the unstabilized solution of alkali or alkaline earth metal hypobromite an aqueous solution of an alkali metal sulfamate having a temperature of at least 50° C. in a quantity to provide a molar ratio of alkali metal sulfamate to alkali or alkaline earth metal hypobromite of from about 0.5 to about 6; and then,
d. Recovering a stabilized aqueous alkali or alkaline earth metal hypobromite solution.

In this embodiment, the alkali or alkaline earth metal hypochlorite is selected from the group consisting of sodium hypochlorite, potassium hypochlorite, magnesium hypochlorite, lithium hypochlorite, and calcium hypochlorite. The bromide ion source is selected from the group consisting of sodium bromide, potassium bromide, lithium bromide, and hydrobromic acid. In a more preferred embodiment, the alkali or alkaline earth metal hypochlorite is sodium hypochlorite, the bromide ion source is sodium bromide, and the alkali or alkaline earth metal hypobromite is sodium hypobromite.

In this embodiment, the ratio of bromide ion source to alkali or alkaline earth metal hypochlorite is preferably about 2.1:1 to ensure the resulting stable oxidizing bromine composition has the desired excess of bromide.

In this embodiment, the pH of the stabilized aqueous alkali or alkaline earth metal hypobromite solution is from about 8 to about 14 and more preferably from about 11 to about 14.

The molar ratio of the alkali metal sulfamate to the sodium hypobromite is preferrably from about 0.5 to about 6, more preferrably from about 0.5 to about 4, and most preferably from about 0.5 to about 2.

In another embodiment, the oxidizing bromine biocidal composition is prepared by mixing (a) bromine chloride or bromine with (b) an aqueous solution of alkali metal salt of sulfamic acid, preferably sulfamic acid sodium salt, the solution having a pH of about 7 to about 13.5, preferably about 7 to about 12. The amounts of (a) and (b) used are such that (i) the content of active bromine in the solution is at least 100,000 ppm (wt/wt) and (ii) the molar ratio of nitrogen to active bromine from (a) and (b) is greater than 1 when bromine is used, and greater than 0.93 when bromine chloride is used. It is preferred however, to utilize a molar ratio of nitrogen to active bromine from (a) and (b) that is greater than 1 even when using bromine chloride in the process. In a preferred embodiment the aqueous solution of alkali metal salt of sulfamic acid used is preformed by mixing together in water, (i) sulfamic acid and/or an alkali metal salt of sulfamic acid, and (ii) alkali metal base in proportions such that an aqueous solution of alkali metal salt of sulfamic acid is formed having a pH of at least about 7. If sulfamic acid itself is used as the starting material, it is used initially as a slurry in water with which the alkali metal base is mixed.

When introducing the bromine chloride or bromine into the aqueous solution of alkali metal salt of sulfamic acid, it is desirable to maintain the desired pH of the resulting solution at about 7 or above by also introducing into the solution (continuously or intermittently, as desired) additional alkali metal base, such as by a co-feed of an aqueous solution of alkali metal base.

In this embodiment, the molar ratio of bromine chloride or bromine to the alkali metal salt of sulfamic acid is preferably about 1. To ensure the resulting stable oxidizing bromine composition has the desired excess of bromide, the alkali or alkaline earth metal bromide is added in a molar ratio of at least about 0.1 to the stable oxidizing bromine made from bromine or is added in a molar ratio of at least about 1.1 to the stable oxidizing bromine solution made from bromine chloride.

A preferred bromine source is bromine chloride.

Alternatively, excess bromide may be added to a system being treated with a conventional stable oxidizing bromine composition prepared, for example, according to the methods described in U.S. Pat. Nos. 5,683,654, 6,068,861, 6,156,229, 6,270,722, 6,423,267 and 6,669,904. In instances where excess bromide source is added to a treated system, the bromide source is added in an amount sufficient to result in a molar ratio of bromide source to stable oxidizing bromine compound in the system of at least about 25.

Accordingly, in another aspect, this invention is a stable oxidizing bromine biocidal composition comprising at least one stable oxidizing bromine compound that is prepared by reacting a first bromide source, oxidant and halogen stabilizer to form a first oxidizing bromine biocidal composition and then adding a sufficient excess of a second bromide source to form an oxidizing bromine biocidal composition having a molar ratio of total bromide source to stable oxidizing bromine compound of at least about 25 in the treated aqueous system.

In a preferred aspect, the second bromide source is an alkali or alkaline earth metal bromide.

In another preferred aspect, the molar ratio of total bromide source to stable oxidizing bromine compound is about 25 to about 2,000.

Certain native waters may have a sufficient concentration of one or more bromide sources, such that a composition having the desired excess of bromide source may be simply prepared by adding the first oxidizing bromine biocidal composition to the native water. For example, sea water typically contains about 60 to about 80 ppm of bromide which is more than adequate to result in the desired ratio of total bromide source to stable oxidizing bromine compound.

Accordingly, in another aspect, this invention is a method of preparing a stable oxidizing bromine biocidal composition comprising reacting a first bromide source, oxidant and halogen stabilizer to form a first oxidizing bromine biocidal composition and then adding the first oxidizing bromine biocidal composition to water having a sufficient concentration of a second bromide source to form an oxidizing bromine biocidal composition having a molar ratio of total bromide source to stable oxidizing bromine compound of at least about 25.

In a preferred aspect of this invention, the water is seawater.

The stabilized bromine solutions which are prepared in accordance with this invention may be used in a wide variety of commercial applications. These applications include, but are not limited to, the use of the stabilized bromine solution: (1) as the bleaching agent in a method for the laundering of soiled garments in which the soiled garments are washed in an aqueous media containing a detergent and a bleaching agent; (2) as the oxidizing agent in a method for the manufacture of cellulosic materials in which cellulosic fibers are bleached; (3) as the oxidizing and biocidal agent in a method for the control of biofouling in a recreational water system in which an oxidizing and biocidal agent is added to control biofouling; (4) as the oxidizing and biocidal agent in a method for the control of biofouling on a hard surface in which an oxidizing and biocidal agent is applied to the surface to control biofouling on the surface; (5) in a method for the control of biofouling occurring on the surfaces of equipment in contact with produced oil field waters; (6) in a method for controlling biofouling in an aqueous system; (7) in a method for controlling biofouling in pulp and paper manufacturing process water and process chemicals; and (8) in a method for controlling microbial growth in an aqueous stream used for transporting or processing food products and on food surfaces and equipment surfaces that come in contact with the aqueous stream.

In another embodiment, the invention is a method of preventing biofouling on the surfaces of equipment in contact with an industrial water system comprising adding an effective biofouling controlling amount of a stabilized bromine solution according to this invention to the water system.

The types of industrial water systems in which the stabilized bromine solution may be used to prevent biofouling include, but are not limited to, cooling water systems, sweetwater systems, gas scrubber systems, air washer systems, evaporative condensers, pasteurizers, produce sanitizer streams, fire protection water systems and heat exchanger tubes.

It is preferred that the amount of stabilized bromine solution which is added to the industrial water system be in the range of about 0.1 ppm to about 2000 ppm and preferably in the range of about 0.5 ppm to about 500 ppm, based on available bromine concentration. The stabilized bromine solution can be added to the water system by any conventional method, i.e., by slug, intermittently or continuously.

The foregoing may be better understood by reference to the following Examples, which are presented for purposes of illustration and are not intended to limit the scope of this invention.

### EXAMPLE 1

An antibacterial study is performed in synthetic cooling water (pH 8.2) containing 1.9×10⁷ CFU/ml cooling water mixed bacteria. Bacterial cell culture is grown in 0.1% tryptic soy broth media overnight, centrifuged, and followed by three washes with phosphate buffer solution (pH7.3). Three stabilized bromine formulation dosages, 1, 2, and 5 ppm (as avail. chlorine), are tested separately with and without extra bromide addition. Bacterial enumeration is done using Aerobic Count Plates Petrifilm (3M, Minneapolis). The biocidal performance result is shown in Table 1.

TABLE 1

| | Test # | | | | | |
|---|---|---|---|---|---|---|
| | 1 | 2 | 3 | 4 | 5 | 6 |
| Stabilized | 1 | 2 | 5 | 1 | 2 | 5 |
| bromine conc. | | | | | | |
| (ppm as Cl₂) | | | | | | |
| Additional | 0 | 0 | 0 | 100 | 100 | 100 |
| bromide conc. | | | | | | |
| (ppm as NaBr) | | | | | | |
| Log reduction at | 2.69 | 6.28 | 5.98 | 6.73 | >7.0 | >7.0 |
| 10 minutes | | | | | | |
| Log reduction at 1 | 5.16 | >7.0 | >7.0 | >7.0 | >7.0 | >7.0 |
| hours | | | | | | |

### EXAMPLE 2

An additional antibacterial study is done to investigate the effect of extra bromide in the case of sulfamate stabilizer cycle-up. The study is done in standard #13 water containing 2.9×10⁷ CFU/ml of cooling water mixed culture bacteria. The test results are shown in Table 2.

TABLE 2

| | Test # | | | | | |
|---|---|---|---|---|---|---|
| | 1 | 2 | 3 | 4 | 5 | 6 |
| | Treatments | | | | | |
| | | | 1 ppm | 1 ppm | 1 ppm | 1 ppm |
| | | | stabilized | stabilized | stabilized | stabilized |
| | | 1 ppm | bromine + | bromine + | bromine + 200 ppm | bromine + 400 ppm |
| | No | stabilized | 100 ppm | 100 ppm | NaBr + 100 ppm | NaBr + 100 ppm |
| Time | treatment | bromine | NaBr | Na-sulfamate | Na-sulfamate | Na-sulfamate |
| (hours) | Log reduction | | | | | |
| 0.17 | 0.0 | 0.10 | 4.86 | −0.26 | −0.09 | 0.00 |
| 1 | 0.0 | 1.42 | 7.46 | 0.03 | 1.76 | 2.18 |
| 4 | −0.2 | 7.46 | 7.46 | 7.46 | 7.46 | 7.46 |

### EXAMPLE 3

Similar experiments are done in phosphate buffer solution (pH 7.5). The initial bacterial concentration is 4.2×10⁵ CFU/ml. The results are summarized Table 3

TABLE 3

| | Test # | | | | | |
|---|---|---|---|---|---|---|
| | 1 | 2 | 3 | 4 | 5 | 6 |
| | Treatments | | | | | |
| | | 1 ppm | 1 ppm | 1 ppm | 1 ppm | 1 ppm |
| | 1 ppm | stabilized | stabilized | stabilized | stabilized | stabilized |
| | stabilized | bromine + | bromine + | bromine + | bromine + | bromine + |
| Time | bromine | 25 ppm NaBr | 50 ppm NaBr | 75 ppm NaBr | 100 ppm NaBr | 150 ppm NaBr |
| (minutes) | Log reduction | | | | | |
| 5 | 3.09 | 4.62 | 4.62 | 5.62 | 5.62 | 5.62 |

The data shown in Tables 1-3 demonstrate that additional bromide is able to significantly improve the speed-of-kill of stabilized bromine formulations. The effect is especially profound when bacterial concentration is high or halogen dosage is low.

In addition, use of extra bromide offsets to some degree the negative effect that excess sulfamate stabilizer imparts on biocidal performance of the bromine. This is significant as stabilizer cycle-up can negatively impact halogen biocidal performance when stabilized bromine formulations are used in any system which is running at high holding time index in systems having high halogen demand.

### EXAMPLE 4

In order to make certain that the observed biocidal performance enhancement is not caused by the bromide addition alone, a set of control experiments are conducted. The results shown in Table 4 demonstrate that the level of bromide addition to phosphate buffer is not biocidal to the mixed cooling water bacterial culture. The differences among all the plate count results are well within the detection limit. Therefore, the performance improvement seen in Examples 1-3 is not caused by the increased concentration of sodium bromide alone.

TABLE 4

| | Test # | | | |
|---|---|---|---|---|
| | 1 | 2 | 3 | 4 |
| | Treatments | | | |
| | No | 50 ppm | 100 ppm | 150 ppm |
| | treatment | NaBr | NaBr | NaBr |
| Time | | | | |
| (minutes) | Bacterial counts in CFU/ml | | | |
| 0 | 7.3 × 10⁵ | 7.3 × 10⁵ | 7.3 × 10⁵ | 7.3 × 10⁵ |
| 5 | 5.7 × 10⁵ | 9.3 × 10⁵ | 11.0 × 10⁵ | 8.4 × 10⁵ |
| 10 | 7.4 × 10⁵ | 7.0 × 10⁵ | 8.2 × 10⁵ | 5.2 × 10⁵ |
| 60 | 9.7 × 10⁵ | 8.0 × 10⁵ | 5.4 × 10⁵ | 8.3 × 10⁵ |

In summary, the major advantages of using extra bromide in stabilized bromine applications include noteworthy improvement in biocidal performance, particularly in terms of speed of kill, of stabilized bromine formulations and prevention of hypobromite disproportionation into bromate while maintaining all the stabilization benefits offered by stabilized bromine biocides (residual, efficacy, etc.).

Changes can be made in the composition, operation, and arrangement of the method of the invention described herein without departing from the concept and scope of the invention as defined in the claims.

1. A stable oxidizing bromine biocidal composition comprising at least one stable oxidizing bromine compound that is prepared from at least one oxidizing chemical reagent, at least one bromide source and at least one bromine or halogen stabilizer, wherein the molar ratio of bromide source to stable oxidizing bromine compound is at least about 2.1.

2. The oxidizing bromine biocidal composition of claim 1 wherein bromine or halogen stabilizer is selected from the group consisting of compounds of formula R—NH—R¹, wherein R and R¹ are selected from the group consisting of R²CO, R²SO₂, R²CF₂, R²CHF, H, OH and PO(OH)₂, or a salt thereof, and R² is an alkyl group or an aromatic group, and mixtures thereof.

3. The oxidizing bromine biocidal composition of claim 2 wherein the oxidizing chemical reagent is selected from the group consisting of alkali and alkaline earth metal hypobromite, alkaline and alkaline earth metal bromates, chlorine gas, hypochlorous acid, alkali and alkaline earth metal hypochlorites, chlorite, hydrogen peroxide, persulfate, permanganate, peracetic acid, bromine, bromine chloride and bromate.

4. The oxidizing bromine biocidal composition of claim 3 wherein the bromide source is selected from the group consisting of bromine, alkali and alkaline earth metal bromides, alkali and alkaline earth metal bromates, hydrobromic acid, bromine chloride, and mixtures thereof.

5. The oxidizing bromine biocidal composition of claim 4 wherein the molar ratio of bromide source to stable oxidizing bromine compound is from about 2.5 to about 5.

6. The oxidizing bromine biocidal composition of claim 5 wherein the stabilizer is selected from the group consisting of saccharine, urea, thiourea, creatinine, cyanuric acids, alkyl hydantoins, monoethanolamine, diethanolamine, organic sulfonamides, biuret, sulfamic acid, organic sulfamates, melamine and ammonia.

7. The oxidizing bromine biocidal composition of claim 6 wherein the molar ratio of bromide source to stable oxidizing bromine compound is from about 2.5 to about 3.

8. A method of preparing the oxidizing bromine biocidal composition of claim 1 comprising reacting the oxidant and halogen stabilizer and a sufficient excess of bromide source to form a composition having a molar ratio of bromide source to stable oxidizing bromine compound of at least about 2.1.

9. A stable oxidizing bromine biocidal composition comprising at least one stable oxidizing bromine compound that is prepared from at least one oxidizing chemical reagent, at least one bromide source and at least one bromine or halogen stabilizer, wherein the molar ratio of bromide source to stable oxidizing bromine compound is at least about 25.

10. A method of preparing the stable oxidizing bromine biocidal composition of claim 9 comprising reacting a first bromide source, oxidant and halogen stabilizer to form a first oxidizing bromine biocidal composition and then adding a sufficient excess of a second bromide source to form an oxidizing bromine biocidal composition having a molar ratio of total bromide source to stable oxidizing bromine compound of at least about 25.

11. The method of claim 10 wherein the second bromide source is an alkali or alkaline earth metal bromide.

12. The method of claim 11 wherein the molar ratio of total bromide source to stable oxidizing bromine compound is about 25 to about 2,000.

13. A method of preparing the stable oxidizing bromine biocidal composition of claim 9 comprising reacting a first bromide source, oxidant and halogen stabilizer to form a first oxidizing bromine biocidal composition and then adding the first oxidizing bromine biocidal composition to water having a sufficient concentration of a second bromide source to form an oxidizing bromine biocidal composition having a molar ratio of total bromide source to stable oxidizing bromine compound of at least about 25.

14. The method of claim 13 wherein the first oxidizing bromine biocidal composition is added to sea water.

15. In a method for the laundering of soiled garments in which the soiled garments are washed in an aqueous media containing a detergent and a bleaching agent, the improvement comprising using as the bleaching agent the oxidizing bromine biocidal composition of claim 1.

16. In a method for the manufacture of cellulosic materials in which cellulosic fibers are bleached with an oxidizing agent, the improvement comprising using as the oxidizing agent the oxidizing bromine biocidal composition of claim 1.

17. In a method for the control of biofouling in a recreational water system in which an oxidizing and biocidal agent is added to control biofouling, the improvement comprising using as the oxidizing and biocidal agent the oxidizing bromine biocidal composition of claim 1.

18. In a method for the control of biofouling on a hard surface in which an oxidizing and biocidal agent is applied to the surface to control biofouling on the surface, the improvement comprising using as the oxidizing and biocidal agent the oxidizing bromine biocidal composition of claim 1.

19. In a method for the control of biofouling occurring on the surfaces of equipment in contact with produced oil field waters, the improvement comprising adding to the produced oil field waters an effective biofouling controlling amount of the oxidizing bromine biocidal composition of claim 1.

20. A method of controlling biofouling in an aqueous system which comprises adding to the aqueous system an effective, biofouling controlling amount of the oxidizing bromine biocidal composition of claim 1.

21. The method of claim 20 wherein the industrial water system is selected from the group consisting of a cooling water system, sweetwater system, gas scrubber system, air washer system, evaporative condenser, pasteurizer, produce sanitizer stream, fire protection water system and heat exchanger tube.

22. The method of claim 20 wherein the oxidizing bromine biocidal composition is added in an amount sufficient to maintain about 25 to about 200 ppm of bromide in the industrial water system.

23. The method of claim 20 wherein the oxidizing bromine biocidal composition is added in an amount sufficient to maintain about 35 to about 150 ppm of bromide in the industrial water system.

24. The method of claim 20 wherein the oxidizing bromine biocidal composition is added in an amount sufficient to maintain about 50 to about 100 ppm of bromide in the industrial water system.
